(19) United States
(12) Patent Application Publication
(10) Pub. No.: US 2007/0108311 A1
(43) Pub. Date: May 17, 2007

(54) Aspiration-type spray system

(75) Inventor: Takeo Matsumoto, Ageo-shi (JP)

Correspondence Address: BIRCH STEWART KOLASCH & BIRCH, PO BOX 747, FALLS CHURCH, VA, 22040-0747, US

(73) Assignee: KYOWA INDUSTRIAL CO., LTD.

(21) Appl. No.: 11/588,278

(22) Filed: Oct. 27, 2006

(30) Foreign Application Priority Data
Nov. 16, 2005 (JP) 2005-331856

(51) Int. Cl. A62C 13/62; F23D 14/28; A61M 11/06; B05B 9/04

(52) U.S. Cl. 239/302; 239/337; 239/338; 239/373

(57) ABSTRACT

An aspiration-type spray system includes a spraying mechanism, a gas cylinder, and a chamber for containing fluid to be sprayed. The spraying mechanism aspirates and sprays the fluid contained in the chamber by spouting a gas from the gas cylinder. The spraying mechanism includes a cylinder-connecting member connected to the stem of the gas cylinder, a fluid-connecting member connected to the fluid chamber, and an actuator for hand-operated spraying. An open/close valve is provided in the fluid-connecting member, and the operation of the actuator not only opens the open/close valve of the fluid-connecting member but presses the stem.

### BACKGROUND OF THE INVENTION

1. Field of the Invention

The present invention relates to an aspiration-type spray system.

2. Description of the Background Art

Conventionally, spray systems of fluid are roughly classified into three types. A first type, which is represented by an aerosol, is configured so that propellant and fluid to be sprayed are contained in a pressurized container, and so that the propellant as well as the fluid is sprayed in the atomized or foamy state. A second type has a dual-container structure in which an inner container is a soft one that contains fluid to be sprayed, and an outer container includes a high-pressure gas so as to apply pressure on the inner container and spray the fluid. A third type, aspiration-type spray system comprised of a gas cylinder connected to a spraying mechanism and a chamber that contains fluid to be sprayed and is also connected to the spraying mechanism but separately from the gas cylinder, sprays the fluid after aspirating it from the chamber as spouting of the contents from the gas cylinder. The spray system of the third type has the following advantages. Since the fluid and the propellant are contained in different containers, compatibility between them or degeneration of the fluid due to the propellant does not have to be considered. In addition, since the chamber does not have to be a pressure-resistant container, the material and design of the spray system may be selected from a wide range of them. However, the third-type spray system is not in fact as popular as the first type. The present invention provides an improved spray system of the third type so as to make effective use of the spray system.

Conventionally, aspiration-type spray systems have been proposed as disclosed in the following Patent Documents 1 to 3.

Patent Document 1: Japanese Unexamined Patent Application published under No. H10-305243 (in 1998).

Patent Document 2: Japanese Unexamined Patent Application published under No. H11-169759 (see FIG. 3) (in 1999).

Patent Document 3: Japanese Examined Utility Model Application published under No. H07-53734 (in 1995).

A spray system disclosed in Patent Document 1 is comprised of a gas cylinder and a fluid container, the fluid being paint here, both of which are completely separate, and a spraying mechanism, to which the gas cylinder and the fluid container are attached. In use, the gas cylinder is held and the actuator thereof is depressed by the user so that the fluid is sprayed. Regarding this type of spray system, since the fluid container is situated in front of the gas cylinder, if the fluid container is considerably large and heavy, the center of gravity is shifted to the front part of the spray system in user's hand, or the fluid container could get in the way, resulting in poor usability.

A spray system disclosed in Patent Document 2 has a fluid-containing chamber provided with a spraying mechanism (aerosol head) of an aerosol serving as a gas cylinder. Regarding this type of spray system, since the aerosol head limits the fluid chamber in size, if the aerosol head is forcibly made large, the upper part of the aerosol weighs too much, resulting in poor usability and vertical instability.

A spray system disclosed in Patent Document 3 is provided with a fluid container under a gas cylinder. Regarding this type of spray system, since a tube connecting the fluid container to a spraying mechanism has to reach the lower part of the gas cylinder by way of a lateral side of the cylinder, resulting in complicated structure of the spraying system.

Further, each of the spray systems disclosed in Patent Documents 1 to 3 does not include means for closing the fluid container, resulting in possibility of leaking out of the fluid from the fluid container in transport.

### SUMMARY OF THE INVENTION

It is, therefore, an object of the present invention to provide an aspiration-type spray system including comprising a spraying mechanism, a gas cylinder connected to the spraying mechanism, and a chamber containing fluid to be sprayed and connected to the spraying mechanism separately from the gas cylinder. The spraying mechanism aspirates and sprays the fluid contained in the chamber by spouting gas from the gas cylinder. The spray system is capable of effectively preventing leakage of a fluid from the fluid chamber during transport or the like. It is another object of the present invention to provide a spray system in which it is possible to keep the weight of the spray system in good balance and to facilitate grasping and using the spray system even if a relatively large fluid container is employed as the fluid-containing chamber. It is yet another object of the present invention to provide a spray system capable of connecting the fluid chamber to the spraying mechanism without using a long tube, and facilitating assembling the spray system with a simple configuration. It is still another object of the present invention to provide a spray system capable of easy disposal of a gas left in the gas cylinder.

The aspiration-type spray system is comprised of a spraying mechanism, a gas cylinder connected to the spraying mechanism, and a chamber containing fluid to be sprayed and connected to the spraying mechanism separately from the gas cylinder. The spraying mechanism aspirates and sprays the fluid in the chamber by spouting a gas from the gas cylinder. The gas cylinder includes a stem having a small hole and an open/close valve that is opened by the stem in response to the pressure applied to it. The spraying mechanism includes a cylinder-connecting member connected to the stem of the gas cylinder, a fluid-connecting member connected to the fluid chamber, and an actuator for hand-operated spraying. An open/close valve is provided in the fluid-connecting member, and the operation of the actuator not only opens the open/close valve of the fluid-connecting member but presses the stem.

In another embodiment of the aspiration-type spray system, the spraying mechanism is provided on an upper end of a casing. The casing is cylindrical extending longitudinally, and an inner space of the cylindrical casing serves as a cylinder space that contains the gas cylinder longitudinally. The fluid chamber is formed longitudinally to be arranged laterally with the gas cylinder space. The spraying mechanism includes a connecting member for the gas cylinder and a connecting member for the fluid chamber, provided under the spraying mechanism.

In other embodiment of the aspiration-type spray system, the casing is a dual-wall cylindrical container including an inner circumferential wall and an outer circumferential wall. A space surrounded by the inner circumferential wall constitutes the cylinder space, and a space between the inner circumferential wall and the outer circumferential wall constitutes the fluid chamber.

In a further embodiment of the aspiration-type spray system, the casing is a single-wall cylindrical container including the outer circumferential wall. An inner space surrounded by the outer circumferential wall serves as both the cylinder space and the fluid chamber. The inner space contains the gas cylinder, and the cylinder's outer diameter is smaller than the inner diameter of the inner space. A space between an outer wall of the gas cylinder and the outer circumferential wall of the casing serves as the fluid chamber.

In a further embodiment of the aspiration-type spray system, the gas cylinder includes a stem having a small hole and an open/close valve that is opened by the stem in response to the pressure applied to the stem. The spray system further comprises a bottom formed on the lower end of the cylinder space and a gas disposal unit provided at the bottom. The gas disposal unit discharges the gas left in a used-up gas cylinder into the cylinder space by pressing the gas cylinder against the bottom from outside.

According to the present invention, an open/close valve is provided in the fluid-connecting member. By operating the actuator, the open/close valve of the fluid-connecting member is opened and the stem is pressed. It is thereby possible to effectively prevent leakage of the fluid from the fluid chamber during transport or the like. Further, only by operating the actuator during use of the spray system, the open/close valve is opened to make it possible to smoothly aspirate and spray the fluid (content liquid).

Also, the spraying mechanism is provided on the upper end of the casing, and the casing is cylindrical extending longitudinally. The inner space of the cylindrical casing serves as a cylinder space that contains the gas cylinder longitudinally, and a chamber containing fluid to be sprayed is formed longitudinally to be arranged laterally with the cylinder space. In addition, the spraying mechanism includes a connecting member for the gas cylinder and a connecting member for the fluid chamber, provided the spraying mechanism. Due to this, it is possible to provide the spray system that can keep the weight of the entire spray system in good balance, thus can be easily held by the user even if the fluid chamber is relatively large, and that can ensure high usability.

Furthermore, the fluid chamber can be connected to the spraying mechanism without a long tube, and the spray system can be assembled with a simple configuration.

Moreover, by providing a gas disposal unit that can discharge the gas in the gas cylinder into the cylinder space at the bottom of the casing. The gas in the gas cylinder can be easily discharged during disposal after use of the spray system.

### BRIEF DESCRIPTION OF THE DRAWINGS

FIG. 1(A) is a cross sectional view of a spray system according to a first embodiment of the present invention, and FIG. 1(B) is a front view of principal parts of the spray system shown in FIG. 1(A);

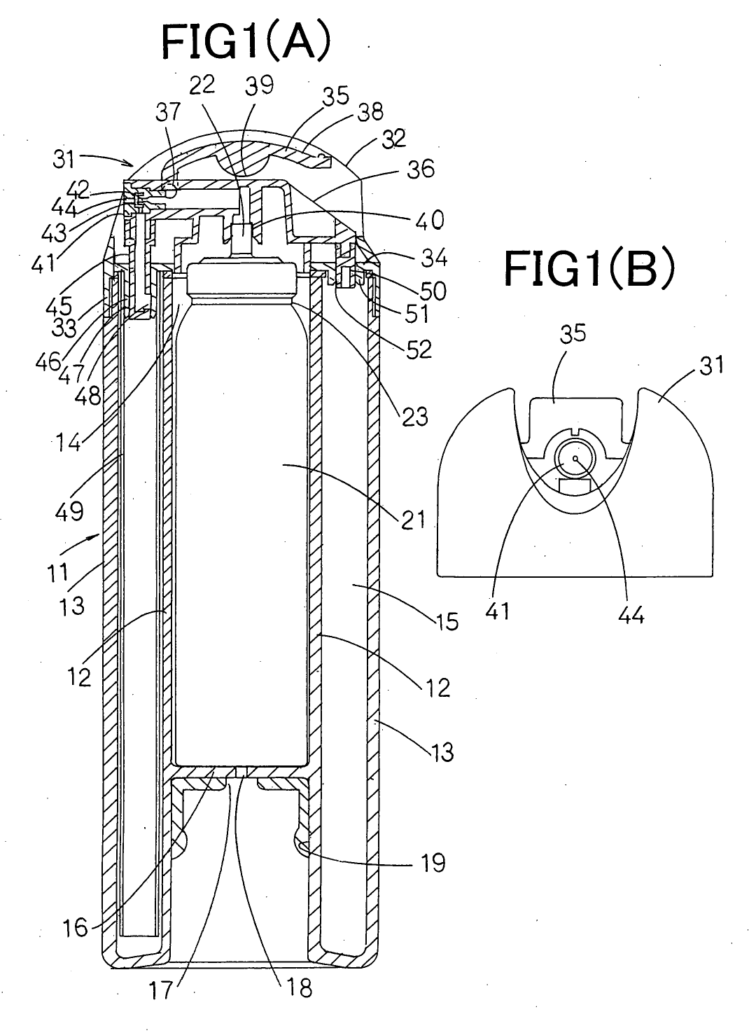

FIG. 2 is a cross sectional view of principal parts of the spray system shown in FIG. 1(A), showing a spraying state of the spray system;

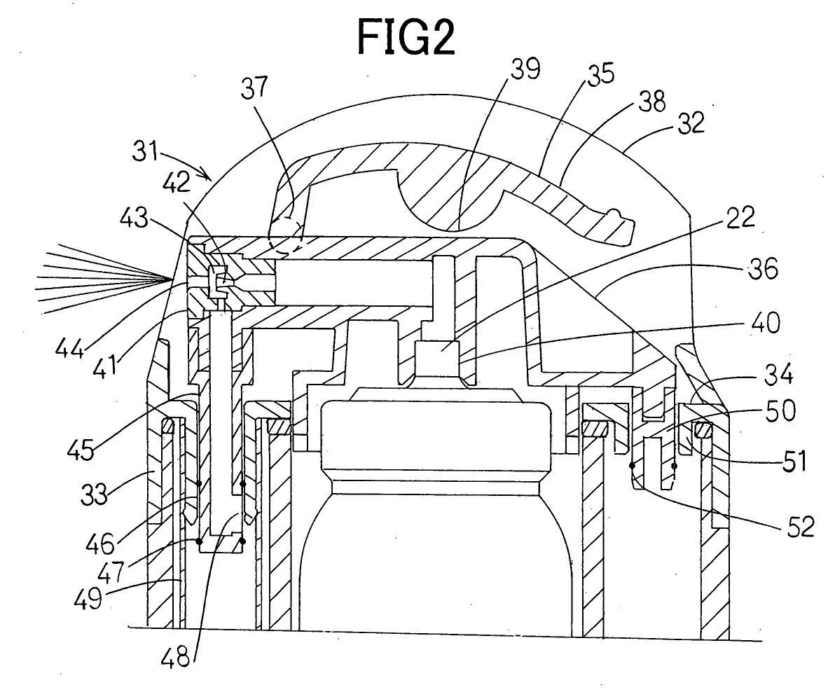

FIG. 3 is a cross sectional view of the spray system, showing a disposal state of the leftover gas of the spray system shown in FIG. 1(A);

FIG. 4 is a cross sectional view of a spray system according to another embodiment of the present invention; and

FIG. 5 is a cross sectional view of principal parts of a spray system according to other embodiment of the present invention;

FIG. 6 is a cross sectional view of a spray system according to a further embodiment of the present invention;

FIG. 7(A) is a cross sectional view of principal parts of the spray system shown in FIG. 6, and FIG. 7(B) is a cross sectional view of principal parts of the spray system shown in FIG. 6, showing a state in use of the spray system.

### DESCRIPTION OF THE PREFERRED EMBODIMENTS

Embodiments of the present invention will be described hereinafter in detail referring to the accompanying drawings.

A spray system in this invention is comprised of a casing 11, a gas cylinder 21 situated in the casing 11, and a spraying mechanism 31 provided on the upper end of the casing 11.

First, the gas cylinder 21 is a container for propellant, such as liquefied gas like DME, LPG, and fluorocarbon, and compressed gas like nitrogen, carbonic acid gas, laughing gas, and air or a mixture of them. The internal pressure of the cylinder may be set equal to an ordinary internal pressure for an aerosol product or a higher pressure than the ordinary, and may be set as desired type as long as the spouting of the gas can aspirate a fluid to be sprayed in a fluid chamber 15, which is described later, and spray it. Further, the gas cylinder 21 may contain chemical fluids used in common with the propellant. The fluid contained in the gas cylinder 21 may be appropriately selected when the fluid can be maintained in the cylinder as well as the propellant, and sprayed together with the propellant. Specifically, the fluid may be the equivalent to be contained in the chamber 15. Being more specific as for a method of use, for example, one constituent component of a two-pack chemical fluid (an exothermic agent, paint, adhesive, hair dye or the like) is contained in the gas cylinder 21 whereas the other constituent component is contained in the chamber 15.

On the upper end of the gas cylinder 21 is provided with a stem 22, in which a small hole (not shown) communicating with the inner space of the cylinder 21 is formed at the upper end thereof. Inside the gas cylinder 21 is provided an open/close valve (not shown) that is opened by the stem 22 in response to the pressure applied thereto. Specifically, the open/close valve is opened when the stem 22 is pressed down or inclined by the pressure, thereby spraying the gas in the cylinder 21 from the small hole of the stem 22. Any of appropriate materials such as metal or synthetic resin may be used for the gas cylinder 21 corresponding to the internal pressure of the propellant. Further, in this embodiment, a neck portion 23 having a smaller diameter than that of the other part of the cylinder 21 is provided near the upper end of the cylinder.

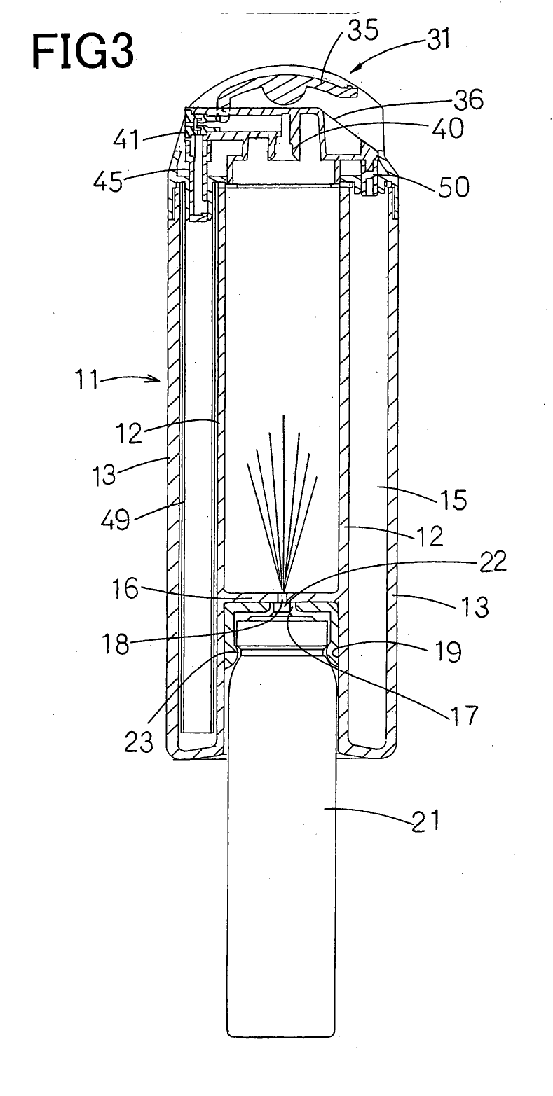

Next, the casing 11 is a dual-wall container that includes an inner circumferential wall 12 and an outer circumferential wall 13. A cylindrical space surrounded by the inner circumferential wall 12 constitutes a space 14 which the gas cylinder 21 sits in. The cylinder space 14, being hollow in cross section, remains unclosed at the upper end until the attachment of the spraying mechanism 31, and has a bottom 16 formed with a plate placed inside the inner circumferential wall 12. The bottom 16 is set at such a position where the stem 22 of the gas cylinder 21 is stably situated with the spraying mechanism 31 when the cylinder 21 sits in the space 14 longitudinally. As shown in FIG. 3, a gas disposal unit is provided at the bottom 16, in which the gas left in a used-up cylinder 21 is discharged into the cylinder space 14. In details, a convex portion 17 is formed outside the bottom 16, which receives the gas cylinder 21, and an opening 18 substantially same in diameter as the small hole of the stem 22 is formed in the convex portion 17. Specifically, the used-up gas cylinder 21 is set fit into the convex portion 17, thus the stem 22 has to be pressed, then the gas left in the cylinder is discharged from the small hole of the stem 22 into the space 14 through the opening 18. This is a safe disposal way of the empty cylinders. In this event, when the casing 11 is provided with a stopper 19 at where the stem 22 is sure to be pressed against the bottom 16, the stopper being able to lock the gas cylinder 21 (at a neck portion 23 in this embodiment), the user do not have to hold the cylinder 21 to discharge the gas while the cylinder 19 is locked by the stopper 19.

Space between the inner and outer circumferential walls 12, 13 of the casing 11 constitutes the aforementioned fluid chamber 15, which is annular in cross section and remains unclosed at the upper end until the attachment of the spraying mechanism 31 in a fluid-tight manner. At the time of closing the fluid chamber 15 fluid-tightly, it is preferable to provide a packing on the upper ends of the inner and outer circumferential walls 12, 13 respectively, or on the lower end of the spraying mechanism 31 so as to keep the space fluid-tight. The chamber 15 has an annular-shaped bottom to be closed fluid-tightly, specifically, since the lower ends of the inner and outer circumferential walls 12, 13 are integrally formed to be coupled to each other, or, in some cases, to be bonded together. The fluid contained in the chamber 15 may be selected freely as long as the fluid can be aspirated by the spouting of the propellant. Examples of the fluid include liquids of daily necessaries, e.g., air-freshener, deodorant, and cleaning agent, and fluids such as cosmetic products, drugs, paint, ink, alcohol, water, as well as powder fluids with properties of fluidity in total such as synthetic resin powder, which is represented by nylon powder, fine powder like silica, hollow microspheres such as shirasu (a kind of silica) balloon, and porous materials.

In the first embodiment, the fluid chamber 15 is longitudinally formed adjacent to the cylinder space 14. More specifically, the cylinder space 14 is positioned inside (at the center) of the fluid chamber 15 that is arranged annularly (cylindrically). A gas cylinder 21 sits in the cylinder space 14 longitudinally. The upper end of the cylinder space 14 is set approximately equal in height to that of the fluid chamber 15. Above the cylinder space 14 and the chamber 15A is provided a spraying mechanism 31.

A spraying mechanism 31 in this embodiment is mounted in a cap 32, which, being in a cap shape and not closed on the bottom, has an attach member 33 on the lower end to be attached to the casing 11. A manner of attachment may be appropriately selected. In this embodiment, the attach member 33 is attached to the upper end of the inner circumferential wall 12 in a detachable fitting or engaging manner. Also, since the upper end of the fluid chamber 15 of the casing 11 is open in this embodiment, a chamber cap 34 is provided on the lower part of the cap 32 to close the fluid chamber 15 at the upper end thereof.

The spraying mechanism 31 is comprised of an actuator 35 for hand-operated spraying, and an acting unit 36 for a spraying movement in response to the manual operation. The actuator 35 has a pivotal supporting portion 37 at the front end, which pivotally supports the actuator to the cap 32, and a depress portion 38 on the surface at the rear end, which the user depresses. On the underside of the actuator between the supporting portion 37 and the depress portion 38 is provided a depresser 39 for depressing the acting unit 36. The user's depressing of the depress portion 38 entirely rotates the actuator 35 downward at the supporting portion 37, whereby the depresser 39 presses down the acting unit 36, which a connecting member 40 is disposed below to be coupled to the stem 22 of the gas cylinder 21. As the acting unit 36 moves down, the stem 22 coupled to the connecting member 40 for cylinder is pressed down before the opening of the open/close valve inside the gas cylinder 21, thereby discharging the propellant that subsequently flows into the connecting member 40. A spray nozzle 41 is attached to the tip end of the connecting member 40 that extends forward. The spray nozzle 41 is comprised of a contracted part 42 that reduces the amount of flow of the propellant in order to accelerate the flow rate of the propellant, a confluent part 43 provided at the end side of said contracted part, and a spray hole 44 that is provided at the end side of said part 43 and sprays a mixture of the propellant and the fluid. The spray hole 44 preferably has a larger inner diameter (channel area) than the contracting part 42 does, more preferably, twice as large or more. Further, the confluent part 43 preferably has a larger inner diameter (channel area) than the spray hole 44 and the contracted part 42 do.

A connecting member 45 communicates with the confluent part 43 at the upper end thereof, and with the fluid chamber 15 at the lower end thereof through a connection hole 46 formed in the chamber cap 34. Further, the connecting member 45 for fluid is provided with an O-ring 47, which functions as a closing valve member, and enables the chamber 15 to be fluid-tightly closed in a usual state. Only when the acting unit 36 is pressed down by the actuator 35, the connecting member 45 communicates with the fluid chamber 15. In details, the connecting member 45 is closed at the lower part thereof, and the O-ring 47 is provided on the closed lower part. Further, a lateral opening 48 is formed on the upper side of the O-ring 47. With the acting unit 36 and the connecting member 45 located at upper positions, the fluid-tightness is kept between the O-ring 47 and the inner wall of the hole 46. With the acting unit 36 and the connecting member 45 located at lower positions, the opening 48 is positioned lower than the connection hole 46, so that the connecting member 45 communicates with the fluid chamber 15. The connection hole 46 is connected with a tube 49 that extends to the bottom of the fluid chamber 15 so as to be able to sufficiently aspirate the fluid present at the bottom of the chamber 15. It is preferable that an opening area of the connecting member 45 with respect to the confluent part 43 is larger than the inner diameter (channel area) of the contracted part 42.

Next, at the rear portion of the acting unit 36 is provided an open/close member 50 for air that is directed downward, and an O-ring 52 that functions as a valve is provided around a lower portion of the member 50. On the other hand, the chamber cap 34 is provided with an air hole 51 into which the open/close member 50 is slidably inserted. With the acting unit 36 located at the upper position, the O-ring 52 keeps airtight between the open/close member 50 and the air hole 51. With the acting unit 36 located at the lower position, the O-ring 52 is located lower than the air hole 51, so that the air can be communicated through a gap between the open/close member 50 and the air hole 51.

With the above-mentioned configuration, as the operation of the actuator 35 makes the acting unit 36 move downward, propellant contained in the gas cylinder 21 is discharged from the small hole of the stem 22, flowing through the cylinder-connecting member 40, the contracted part 42, and the confluent part 43 before being sprayed from the spray hole 44. Concurrently with this, the downward moving of the acting unit 36 opens a valve (O-ring 47) of the connecting member 45, whereby the fluid chamber 15 communicates, through the tube 49 and the connecting member 45, with the confluent part 43. Following the propellant's spraying from the spray hole 44, the internal pressure of the confluent part 43 becomes negative, and the fluid in the chamber 15 is aspirated through the connecting member 45 and the tube 49 before being sprayed, together with the propellant, from the spray hole 44. At this time, with the acting unit 36 moving down, a valve (O-ring 52) of the open/close member 50 is opened, while the internal pressure of the fluid chamber 15 becomes equal to atmospheric pressure, thereby achieving sufficient continuous aspiration and spraying of the fluid.

When the user releases the depressed member 38 of the actuator 35, the actuator 35 returns to the upper position owing to conventional urging means (not shown) provided in the gas cylinder 21 that urges the stem 22 upward, then the spraying of the propellant stops, while the valves of the connecting member 45 and the open/close member 50 are closed, then the fluid returns to be maintained in a fluid-tight and airtight state. As a result, there is no fear of leakage of the fluid in the chamber 15 during transport. In this embodiment, although the actuator 35 is pressed down, a manner of pressing the actuator 35 can be appropriately changed. For example, the actuator 35 can be pressed laterally or lifted up. Further, the detailed structure of the spraying mechanism 31 can be appropriately changed to, for example, the structure of the conventional spray system such as those disclosed in Patent Documents 1 to 3, when the fluid can be sprayed along with the spraying of the propellant. In this manner, setting of the respective upper ends of the gas cylinder 21 and the fluid chamber 15 to reach the upper portion of the casing 11 can achieve a simple mechanism to open and close the respective valves of the connecting member 45 and the open/close member 50 with a single operation of the actuator 35. In particular, providing a valve of the open/close member 50 on the chamber cap 34 can achieve a valve-equipped open/close member 50 in the fluid chamber 15 by simply attaching the cap 32 to the casing 11.

Moreover, the casing 11 and the cap 32 (spraying mechanism 31) may be detachably fitted or engaged to replace a gas cylinder 21 and supply fluid in the fluid chamber 15, let alone fixed in order not to be detached by the user. In disposal of it, as described above, the gas left in the used-up cylinder 21 can be discharged, and only the empty cylinder 21 can be dumped separately. Further, the bottom 16 of the cylinder space 14 can be detachably provided as a sole detachable member. Furthermore, a single fluid chamber 15 is provided in the casing 11 in this embodiment. Alternatively, two or more fluid chambers may be provided by radially partitioning the fluid chamber 15 into a plurality of segments. In this case, two or more connecting members 45 for fluid are correspondingly provided, and multiple types of the fluids can be mixed together at or before the confluent part 43. Or, the fluids can be selectively used with providing of an open/close or swing valve.

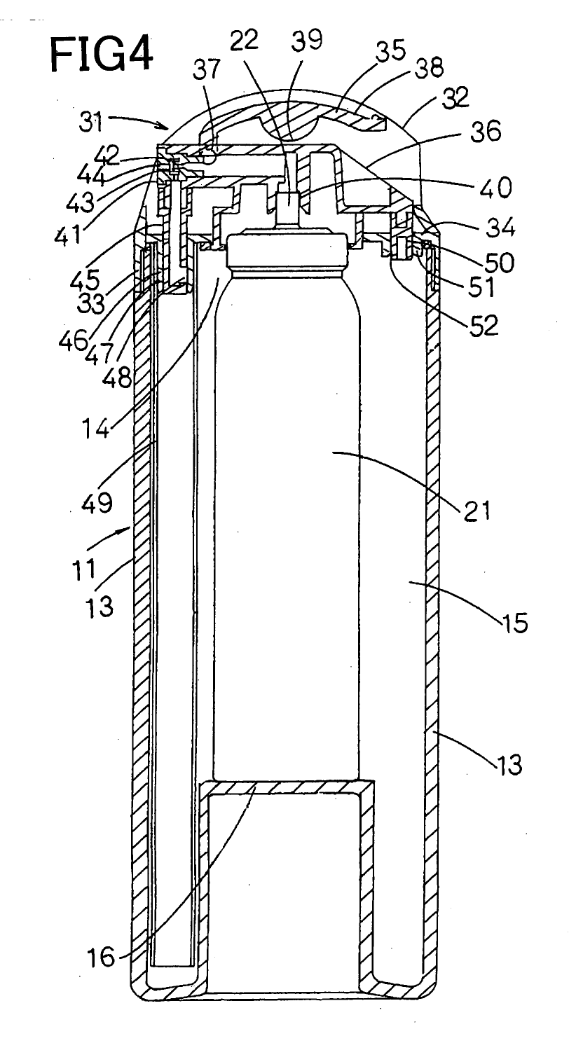

As shown in FIG. 4, the casing 11 can be configured not to provide an inner circumferential wall 12 according to the first embodiment. According to another embodiment of the present invention, the casing 11 is a single-wall cylindrical container with only the outer circumferential wall 13, which constitutes an internal space which serves as both a cylinder space and a fluid-containing space. The outer diameter of the gas cylinder 21 is set smaller than the inner diameter of the outer circumferential wall 13, then the space formed between the outer wall of the gas cylinder 21 and the wall of the casing 11 (outer circumferential wall 13) serves as a fluid chamber 15. It is to be noted that, in order to keep fluid-tight inside the space, the opening 18 in the bottom 16 according to the first embodiment is either not formed or may be formed with an open/close valve. It is further necessary to close the gap between the upper portion of the gas cylinder 21 and that of the outer circumferential wall 13 so as to keep it liquid-tight.

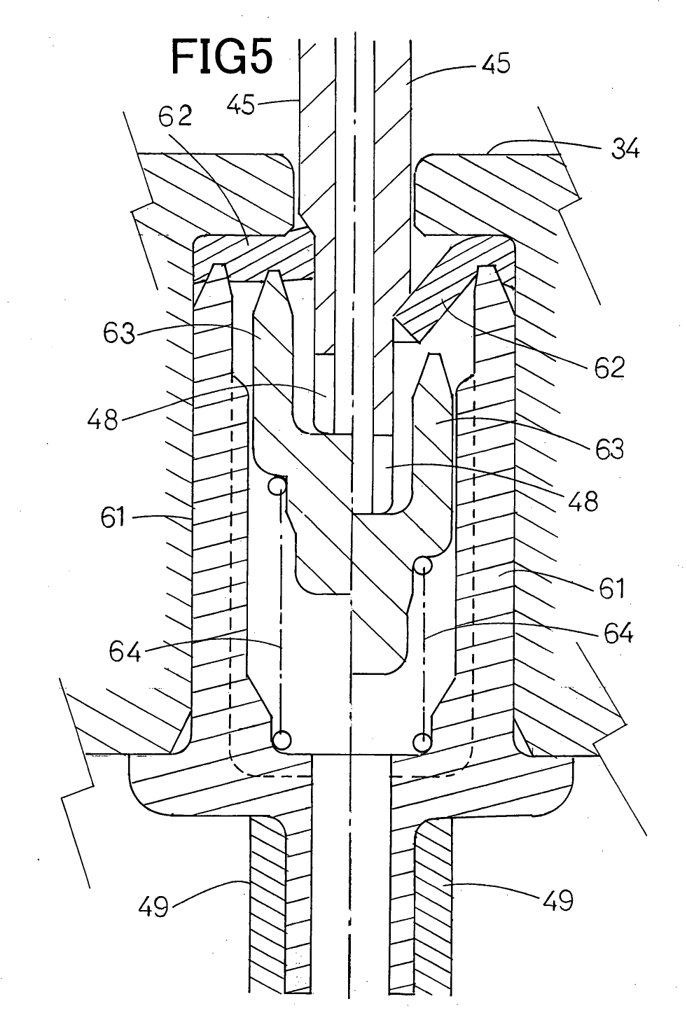

Moreover, the valve structure provided on the connecting member 45 can be variously changed as long as the valve structure is closed in the usual state and can be opened based on the operation of the actuator. As shown in FIG. 5, a valve housing 61 is provided in the chamber cap 34 in a spray system according to other embodiment of the present invention. On the upper end of the valve housing 61 is provided a deformable elastic seal member 62 made of rubber or the like, underside of which is placed a bottomed cylindrical valve 63 that is slidable vertically and is urged upward by a spring 64 or the like to fluid-tightly close the gap between an upper end of the valve 63 and the seal member 62. Similarly to the first embodiment, the tubular connecting member 45 having the opening 48 in the lower end thereof is provided to penetrate the seal member 62 and abut on the bottom of the valve 63 from above. The lower end of the valve housing 61 is unclosed and connected to the tube 49. By so configuring, the fluid-tightness is kept between the upper end of the valve 63 and the seal member 62 in the usual state as shown in the left-half part of FIG. 5. On the other hand, as shown in the right-half part of FIG. 5, with the connecting member 45 pressed down by the operation of the actuator 35, the valve 63 is opened against the urging force of the spring 64, and the fluid can flows into the tube 49, the lower space in the valve housing 61, the space between the valve housing 61 and the valve 63, the upper space in the valve housing 61, the space between the valve 63 and the seal member 62, the inner space of the valve 63, the opening 48, and the connecting member 45 in this order.

The valve structure according to the other embodiment can be used not only as that of the connecting member 45 for fluid but also as that of the open/close member 50 for air.

As described so far, the spray system according to the present invention can be configured to have a similar appearance to that of the well-known aerosol spray, to have a good weight balance, and to ensure high usability. The shape of the casing of the spray system according to the present invention is not limited to a cylindrical one but may be appropriately changed to, for example, a prismatic one.

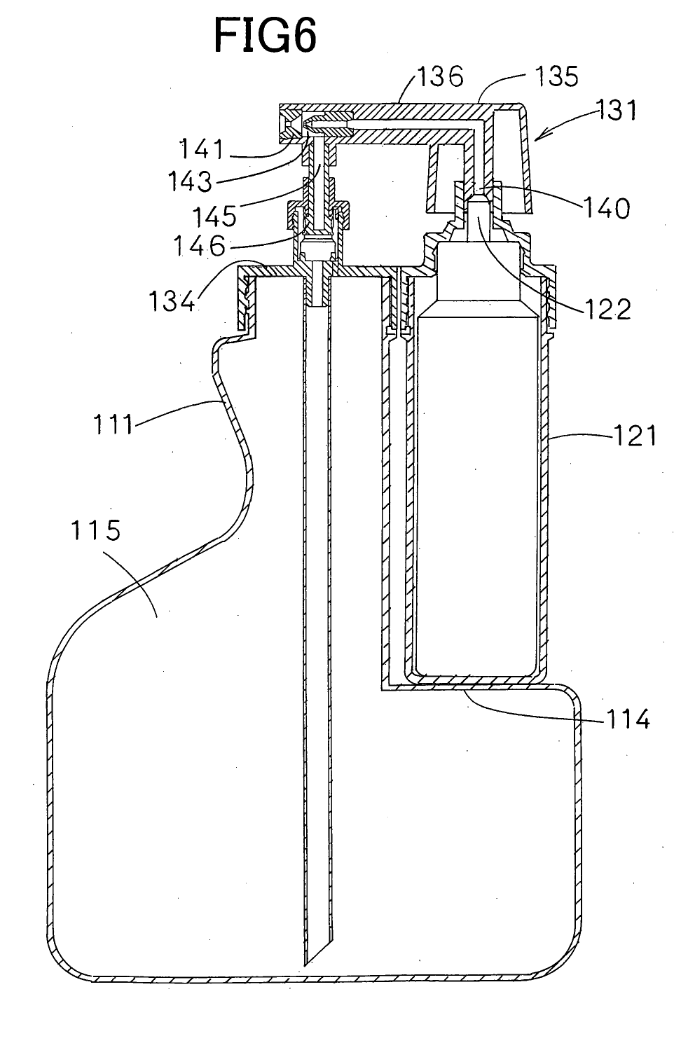

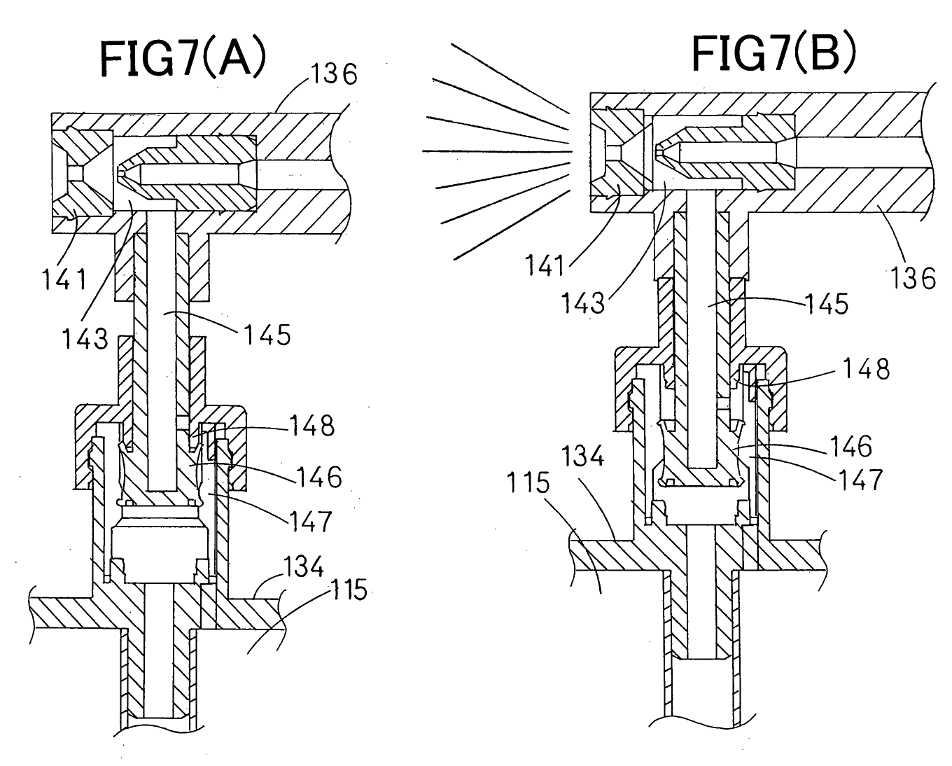

Next, a further embodiment of the present invention will be described hereinafter referring to FIGS. 6 and 7.

Even in this embodiment, a spray system is substantially identical to the aforementioned one on the point of comprising a casing 111, a gas cylinder 121, and a spraying mechanism provided on the upper end of the casing 111. However, it makes a difference that the gas cylinder 121 in this embodiment is provided outside the casing 111, while the gas cylinder 21 in the aforementioned embodiment is provided within the casing 11.

The casing 111 is a sealable container for liquid that is comprised of a single wall. The entirety of the inside of the casing serves as a fluid chamber 115, where, same as the previous embodiments, fluid to be sprayed is contained. The shape of the casing in this embodiment is not limited to the one shown in the figure, but may be appropriately applicable to a cylindrical or prismatic one. The casing 111 in this example is provided with a cylinder space 114 where a gas cylinder 121 is placed. Top ends of the casing 111 and the gas cylinder 121 are disposed at substantially the same height, and a spray mechanism 131 is mounted on the top ends thereof.

The gas cylinder 121 may contain various propellants as the aforementioned does. On the upper end of the gas cylinder 121 is provided with a stem 122, in which a small hole (not shown) communicating with the inner space of the cylinder 121 is formed at the upper end thereof. Inside the gas cylinder 121 is provided an open/close valve (not shown) that is opened by the stem 122 in response to the pressure applied thereto. Specifically, the open/close valve is opened when the stem 122 is pressed down or inclined by the pressure, thereby spraying the gas in the cylinder 121 from the small hole of the stem 122.

The spraying mechanism 131 is comprised of an actuator 135 for hand-operated spraying, and an acting unit 136 for a spraying movement in response to the manual operation. Below the acting unit 136 is disposed a connecting member 140 that is coupled to the stem 122 of the gas cylinder 121. As the acting unit 136 moves down, the stem 122 is pressed down before the opening of the open/close valve inside the gas cylinder 121, thereby discharging the propellant that subsequently flows into the connecting member 140. A spray nozzle 141, which has the same structure in details as the first embodiment, such as including a confluent part 143, is attached to the tip end of the connecting member 140 that extends forward.

As shown in FIG. 7, a connecting member 145 communicates with the confluent part 143 at the upper end thereof, and is provided with an open/close valve 146 at the lower end thereof. A lid 134 for the fluid chamber 115 is formed with a connection chamber 147, where a valve seat 148 is provided. The connecting member 145, the lower end of which is slidably inserted into the connection chamber 147, opens and closes a channel between the open/close valve 146 and the valve seat 148. In details, the fluid chamber 115 is fluid-tightly closed by the open/close valve 146 that contacts the valve seat 148 in a usual state. Only when the acting unit 136 is pressed down, the open/close valve 146 is released from the valve seat 148, thus the connecting member 145 communicates with the fluid chamber 115. Consequently, the propellant is discharged out of the gas cylinder 121, while the internal pressure of the confluent part 143 becomes negative, then the fluid to be sprayed in the fluid chamber 115 is aspirated through the connecting member 145 before being sprayed, together with the propellant, from the spray hole 141. Like the previous example, an open/close member for air, which opens concurrently with the downward moving of the acting unit 136, may be provided.

As described so far, the spray system according to the present invention may have a variety of shapes of the casing 111 and the gas cylinder 121, and be designed freely corresponding to its purpose.

What is claimed is:

1. An aspiration-type spray system comprising a spraying mechanism, a gas cylinder connected to the spraying mechanism, and a chamber containing fluid to be sprayed and connected to the spraying mechanism separately from the gas cylinder, the spraying mechanism aspirating and spraying the fluid in the fluid chamber by spouting a gas from the gas cylinder,
wherein the gas cylinder includes a stem having a small hole and an open/close valve that is opened by the stem in response to the pressure applied thereto,
wherein the spraying mechanism includes a cylinder-connecting member connected to the stem of the gas cylinder, a fluid-connecting member connected to the fluid chamber, and an actuator for hand-operated spraying, wherein an open/close valve is provided in the fluid-connecting member, and wherein the operation of the actuator not only opens the open/close valve of the fluid-connecting member but presses the stem.

2. The aspiration-type spray system according to claim 1, wherein the spraying mechanism is provided on an upper end of a casing, the casing being cylindrical extending longitudinally, an inner space of the cylindrical casing serving as a cylinder space that contains the gas cylinder longitudinally, while the fluid chamber is formed longitudinally to be arranged laterally with the gas cylinder space, and
wherein the spraying mechanism includes a connecting member for the gas cylinder and a connecting member for the fluid chamber, the members being provided under the spraying mechanism.

3. The aspiration-type spray system according to claim 2, wherein the casing is a dual-wall cylindrical container including an inner circumferential wall and an outer circumferential wall, and a space surrounded by the inner circumferential wall constitutes the cylinder space, while a space between the inner and outer circumferential walls constitutes the fluid chamber.

4. The aspiration-type spray system according to claim 2, wherein the casing is a single-wall cylindrical container including an outer circumferential wall, and an inner space surrounded by the outer circumferential wall serves as both the cylinder space and the fluid chamber, the inner space containing the gas cylinder, the outer diameter of which is smaller than the inner diameter of the inner space, while a space between the outer wall of the gas cylinder and the outer circumferential wall of the casing serves as the fluid chamber.

5. The aspiration-type spray system according to claim 2, wherein the gas cylinder includes a stem having a small hole and an open/close valve that is opened by the stem in response to the pressure applied thereto, and
wherein the spray system further comprises a bottom formed on the lower end of the cylinder space and a gas disposal unit provided at the bottom, the gas disposal unit discharging the gas left in the used-up gas cylinder into the cylinder space by pressing the gas cylinder against the bottom from outside.

6. The aspiration-type spray system according to claim 1, wherein the spraying mechanism includes a cylinder-connecting member connected to the stem of the gas cylinder, a fluid-connecting member connected to the fluid chamber, an open/close member for air that introduces air into the fluid chamber, and an actuator for hand-operated spraying, wherein open/close valves are respectively provided in the fluid-connecting member and the open/close member for air, and wherein the operation of the actuator not only opens the open/close valves of the fluid-connecting member and the open/close member for air but presses the stem.
